(19) United States
(12) Patent Application Publication
(10) Pub. No.: US 2020/0343000 A1
(43) Pub. Date: Oct. 29, 2020

(54) MATCHING ENDOSCOPIC PATIENTS WITH MEDICAL PERSONNEL

(71) Applicant: International Business Machines Corporation, Armonk, NY (US)

(72) Inventors: Uri Kartoun, Cambridge, MA (US); Fang Lu, Billerica, MA (US); David Osofsky, Arlington, MA (US); Kenney Ng, Arlington, MA (US)

(21) Appl. No.: 16/393,472

(22) Filed: Apr. 24, 2019

(51) Int. Cl. G16H 80/00; G16H 10/60; G06N 20/00; G06F 16/2457

(52) U.S. Cl. CPC G16H 80/00; G06F 16/2457; G06N 20/00; G16H 10/60

(57) ABSTRACT

Embodiments provide a computer implemented method of identifying a group of medical professionals for performing an endoscopic procedure on a particular patient, including: training a machine learning model with a plurality of electronic medical records of different patients having a history of one or more endoscopic procedures, wherein each electronic medical record includes a group of medical professionals performing the one or more endoscopic procedures, and recovery time of each endoscopic procedure; receiving an electronic medical record of a new patient intending to have an endoscopic procedure; calculating a score for each medical professional in a medical organization representing a level of match between the new patient and each medical professional; and identifying a group of medical professionals for performing the endoscopic procedure on the new patient.

### TECHNICAL FIELD

The present disclosure relates generally to a system, method, and computer program product that can identify a group of medical professionals including, e.g., a physician, a technician, and a nurse, who are most suitable for an endoscopic patient.

### BACKGROUND

For patients who attended an endoscopic procedure, there is a substantial variability in length of recovery time (referred to as “waiting time”) after performing the endoscopic procedure. For example, the recovery time can be in a range of a few minutes to several hours. An unexpectedly long recovery time generally indicates a less successful procedure. A recent study reports that the most predictive factor associated with the unexpectedly long recovery time is a group of medical professionals performing the endoscopic procedure. The group of medical professionals can include one or more physicians, one or more technicians, and one or more nurses.

Thus, it is desired to identify a best matched group of medical professionals for a patient who needs to attend an endoscopic procedure, so as to minimize a possibility of long recovery time.

### SUMMARY

Embodiments provide a computer implemented method in a data processing system comprising a processor and a memory comprising instructions, which are executed by the processor to cause the processor to implement the method of identifying a group of medical professionals for performing an endoscopic procedure on a particular patient, the method comprising: training, by the processor, a machine learning model with a plurality of electronic medical records of different patients having a history of one or more endoscopic procedures, wherein each electronic medical record includes a group of medical professionals performing the one or more endoscopic procedures, and recovery time of each endoscopic procedure, wherein the group of medical professionals include at least one physician, at least one technician, and at least one nurse; receiving, by the machine learning model, an electronic medical record of a new patient intending to have an endoscopic procedure; calculating, by the machine learning model, a score for each medical professional in a medical organization representing a level of match between the new patient and each medical professional, wherein all the medical professionals in the medical organization are divided into three categories of physician, technician, and nurse; and identifying, by the machine learning model, a group of medical professionals for performing the endoscopic procedure on the new patient, wherein the group of medical professionals include a physician having the highest score in a category of physician, a technician having the highest score in a category of technician, and a nurse having the highest score in a category of nurse.

Embodiments further provide a computer implemented method, further comprising: calculating, by the machine learning model, the score through one or more of machine learning algorithms including logistic regression, Bipartite Ranking, k-partite Ranking, Ranking with Real-Valued Labels, General Instance Ranking, Ranking SVM, RankBoost, and RankNet.

Embodiments further provide a computer implemented method, further comprising: if there was a prior patient having short recovery time after the one or more endoscopic procedures performed by the group of the medical professionals including more than one nurse, and the prior patient has substantially the same medical condition as that of the new patient, adding, by the machine learning model, an additional nurse into the group of medical professionals for performing the endoscopic procedure on the new patient.

Embodiments further provide a computer implemented method, further comprising: if there was a prior patient having short recovery time after the one or more endoscopic procedures performed by the group of the medical professionals including more than one technician, and the prior patient has substantially the same medical condition as that of the new patient, adding, by the machine learning model, an additional technician into the group of medical professionals for performing the endoscopic procedure on the new patient.

Embodiments further provide a computer implemented method, further comprising: assigning, by the machine learning model, a more experienced group of medical professionals if the new patient has a plurality of diseases or at least one comorbidity.

Embodiments further provide a computer implemented method, wherein the plurality of diseases includes at least two chronic conditions including type-2 diabetes, hypertension, hyperlipidemia, arthritis, and depression.

Embodiments further provide a computer implemented method, wherein each electronic medical record includes at least one of characteristics including demographic details, allergies, diagnoses, vital signs, laboratory tests, clinical narrative notes, regular physical exams, pathology reports, discharge summaries, radiology reports, cardiology reports, encounters, comorbidities, endoscopic procedures, other procedures, and medications.

In another illustrative embodiment, a computer program product comprising a computer usable or readable medium having a computer readable program is provided. The computer readable program, when executed on a processor, causes the processor to perform various ones of, and combinations of, the operations outlined above with regard to the method illustrative embodiment.

In yet another illustrative embodiment, a system is provided. The system may comprise a processor configured to perform various ones of, and combinations of, the operations outlined above with regard to the method illustrative embodiments.

Additional features and advantages of this disclosure will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings. For the purpose of illustrating the invention, there is shown in the drawing embodiments that are presently preferred, it being understood, however, that the invention is not limited to the specific instrumentalities disclosed. Included in the drawings are the following Figures:

FIG. 1 depicts a block diagram of an example endoscopic patient-medical professional match system 100, according to embodiments herein;

FIG. 2 is an example flowchart illustrating a method 200 of identifying a group of medical professionals, according to embodiments herein;

FIG. 3 is an example table 300 illustrating three lists of ranked medical professionals output by a machine learning model, according to embodiments herein; and

FIG. 4 is a block diagram of an example data processing system 400 in which aspects of the illustrative embodiments may be implemented.

### DETAILED DESCRIPTION OF EXEMPLARY EMBODIMENTS

Embodiments of the present invention may be a system, a method, and/or a computer program product. The computer program product may include a computer readable storage medium (or media) having computer readable program instructions thereon for causing a processor to carry out aspects of the present invention.

In an embodiment, a system, method, and computer program product can provide a precise match between a patient who intends to attend an endoscopic procedure and a group of medical professionals who can perform the endoscopic procedure.

In another embodiment, the system, method, and computer program product can propose adding another medical professional (e.g., an additional nurse) to help with an endoscopic procedure, so as to achieve a shorter recovery time for a patient. This is possible if any patients having similar medical conditions to that of the current patient benefitted from adding another medical professional in prior endoscopic procedures.

The system, method, and computer program product collect electronic medical records (EMRs) of a large population of patients who previously attended endoscopic procedures. In an embodiment, each EMR can include recovery time of a particular patient, a group of medical professionals who performed an endoscopic procedure, and all possible details of the particular patient. The collected EMRs are used to train a machine learning model. An EMR of a new patient who intends to attend the endoscopic procedure is input into an trained machine learning model, which then outputs a specific group of medical professionals including at least one physician, at least one technician, and at least one nurse, who could fit better to the patient, thereby resulting in a decreased recovery time for the upcoming endoscopic procedure.

In an embodiment, the possible details of a particular patient may include any information regarding the patient's medical condition and socioeconomic status extracted from the EMR of the particular patient. Such information includes a large collection of lab observations (e.g., creatinine, sodium, hemoglobin), medications, current and past comorbidities, vital signs, age, gender, ethnicity, education level, insurance status, and risk scores (such as Model For End-Stage Liver Disease (MELD) score, MELD-Na score considering a sodium element, and MELD-Plus score including nine variables as effective predictors for 90-day mortality after a discharge from a cirrhosis-related admission), etc. In another embodiment, additional possible details may be directly related to any procedures the particular patient has attended, such as average recovery time of previous procedures, the number of previous procedures, and the number of successful procedures vs. the number of unsuccessful procedures, etc.

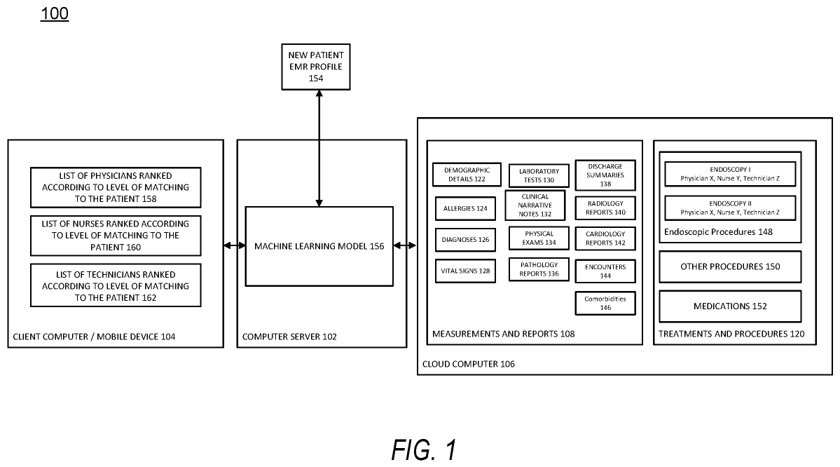

FIG. 1 depicts a block diagram of an example endoscopic patient-medical professional match system 100, according to embodiments herein. As shown in FIG. 1, in an embodiment, the computer server 102, the client computer 104, and the cloud computer 106 are an example of a computer, such as a server or client, in which computer usable code or instructions implementing the process for illustrative embodiments of the present invention are located. In one embodiment, the computer server 102, the client computer 104, and the cloud computer 106 represent a computing device, which implements an operating system. In some embodiments, the operating system can be Windows, Unix system or Unix-like operating systems, such as AIX, A/UX, HP-UX, IRIX, Linux, Minix, Ultrix, Xenix, Xinu, XNU, and the like.

As shown in FIG. 1, in an embodiment, a large collection of EMRs for patients having a history of endoscopic procedures are stored on the cloud computer 106 or a remote server. In an embodiment, EMR includes measurements and reports 108, and treatments and procedures 120. The measurements and reports 108 can include but not limited to, demographic details 122 including, e.g., age, gender, ethnicity, marital status, and insurance type, etc.; allergies 124 illustrating symptoms and substances which results in allergic reactions; diagnoses 126 illustrating diagnosis results of diseases; vital signs 128 including information on respiratory, temperature, as well as blood pressure values; laboratory tests 130 including, e.g., urine tests, blood tests, stool tests, etc.; clinical narrative notes 132 illustrating specific clinical events or situation; regular physical exams 134; pathology reports 136 which contains the diagnosis determined by examining cells and tissues under a microscope; discharge summaries 138 for hospital discharge; radiology reports 140 which are written communications between a radiologist interpreting an imaging study and a physician who requested an imaging scan; cardiology reports 142; encounters 144 including an “inpatient encounter,” an “outpatient encounter,” a “telephone encounter,” an “email encounter,” an “emergency room visit,” a “home visit,” etc.; and comorbidities 146 including, e.g., liver, cardiovascular, or respiratory illnesses, etc.

The treatments and procedures 120 include but not limited to, endoscopic procedures 148 related information, e.g., medical professionals who performed the endoscopic procedures 148 and recovery time; other procedures 150 including surgeries (e.g., transplantation, heart catheterization, etc.), and immunizations; and medications 152.

As shown in FIG. 1, the machine learning model 156 is running on the computer server 102, which is wiredly or wirelessly connected to the cloud computer 106 and the client computer 104 (or a mobile device, such as a mobile phone, a tablet, a personal digital assistant, etc.). Machine learning, also referred to as “artificial intelligence,” is a group of techniques that allows computers to not only process data faster than humans, but also to process data more intelligently. The machine learning model 156 allows a computer to observe large collections of data elements and provide accurate predictions on the occurrence of future events. The machine learning model 156 can be either supervised machine learning model or unsupervised machine learning model. Examples for machine learning algorithms include but not limited to: deep learning, neural network, logistic regression, support vector machines, reinforcement learning, etc. The machine learning model 156 is trained by a large collection of structured and/or unstructured EMRs of different patients stored on the cloud computer 106.

Further as illustrated in FIG. 1, the EMR 154 of a new patient is input into the machine learning model 156 trained by a large number of EMRs of different patients who experienced endoscopic procedures. A list 158 of physicians ranked according to level of matching to the new patient, a list 160 of nurses ranked according to level of matching to the new patient, and a list 162 of technicians ranked according to level of matching to the new patient are output on the client computer/mobile device 104. In an embodiment, a physician, a nurse, and a technician having the highest score can be selected into a group of medical professionals who are going to perform an endoscopic procedure for the new patient.

There are various approaches to calculate a score that represents the level of match between a patient and a medical professional. A typical approach is probabilistic classifier (e.g., logistic regression) and uses an equation to calculate a probability for a certain scenario (e.g., what is the probability for short recovery time (e.g., less than 30 mins) if a certain medical professional is assigned to a patient). Other approaches, for example, include algorithms such as “Bipartite Ranking,” “k-partite Ranking,” “Ranking with Real-Valued Labels,” “General Instance Ranking,” “Ranking SVM,” “RankBoost,” and “RankNet,” etc.

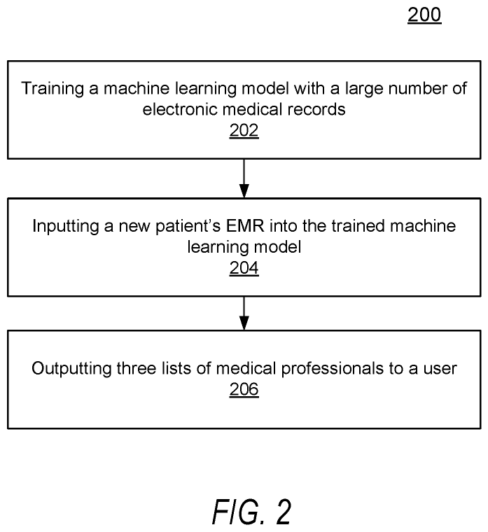

FIG. 2 is an example flowchart illustrating a method 200 of identifying a group of medical professionals, according to embodiments herein. At step 202, the machine learning model 156 is trained with a large number of EMRs of different patients. Each EMR includes the whole medical record of a particular patient, particularly including all the information related to one or more endoscopic procedures attended by the particular patient. At step 204, a new patient's EMR is input into the trained machine learning model 156.

At step 206, three lists of medical professionals are output from the machine learning model 156 to a user, e.g., a chief physician. Each list represents a different category of medical professionals, e.g., physicians, nurses and technicians. The chief physician can select the top ranked medical professional in each category. In another embodiment, only the suggested medical professionals, i.e., the top ranked medical professional in each category, are directly proposed to the user, without showing the whole lists.

In an embodiment, a more complex patient may be assigned more experienced medical professionals, which results in a reduced recovery time. A complex patient is an individual who suffers from multiple diseases or at least one comorbidity (e.g., multiple sclerosis). For example, a patient with several common chronic conditions, such as type-2 diabetes, hypertension, hyperlipidemia, arthritis, and major depression, etc., can be identified as a complex patient. A patient with more than a predefined number of (e.g., three) chronic conditions is considered as being more complex. Further, a complex patient typically needs to take a multiple number of (e.g., 10) medication types per day, and thus is more difficult to manage considering potential side effects and lack of adherence to medications.

An experienced medical professional has many years of experience. For instance, a nurse having 20 years of experience who has participated in more than 1000 endoscopic procedures is considered as being more experienced than a nurse who has only participated in less than 100 endoscopic procedures. A newly hired technician who has participated in endoscopic procedures in other US hospitals is considered as being more experienced than a newly hired technician who only participated in endoscopic procedures in foreign hospitals (i.e., non-US hospitals).

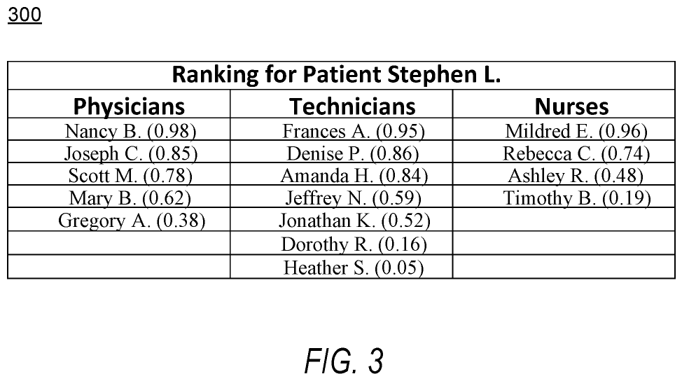

In an example, there are 16 medical professionals, i.e., 5 physicians, 7 technicians, and 4 nurses, in charge of endoscopic procedures in a particular medical organization, e.g., a hospital, a clinic, and the like. The EMRs of patients who attended endoscopic procedures in this hospital are used to train a machine learning model 156. A new patient's EMR is input into the trained machine learning model 156, and each medical professional is provided with a score (e.g., in a range of 0 to 1, wherein the higher the score, the better the match between this new patient and a particular medical professional) based on the EMR of this new patient. For example, as shown in table 300 of FIG. 3, for a new patient Stephen L., a score will be calculated for each of 16 medical professionals in three different categories, i.e., “physician,” “technician,” and “nurse.” In each category, the medical professionals are sorted or ranked in a descending order according to their level of match relative to Stephen L. Stephen L. is expected to have the shortest recovery time if Nancy B. (a physician), Frances A. (a technician), and Mildred E. (a nurse) perform the endoscopic procedure for him.

In an embodiment, one or more additional medical professionals can be proposed in addition to the top ranked medical professional in each category. For example, in addition to the top ranked physician Nancy B., the top ranked technician Frances A., and the top ranked nurse Mildred E., the second ranked nurse Rebecca C. can also be proposed for the upcoming endoscopic procedure. This is proposed if there were patients benefitting from having one or more additional medical professionals perform an endoscopic procedure, and these former patients have a similar medical condition or/and socioeconomic details to that of the new patient Stephen L.

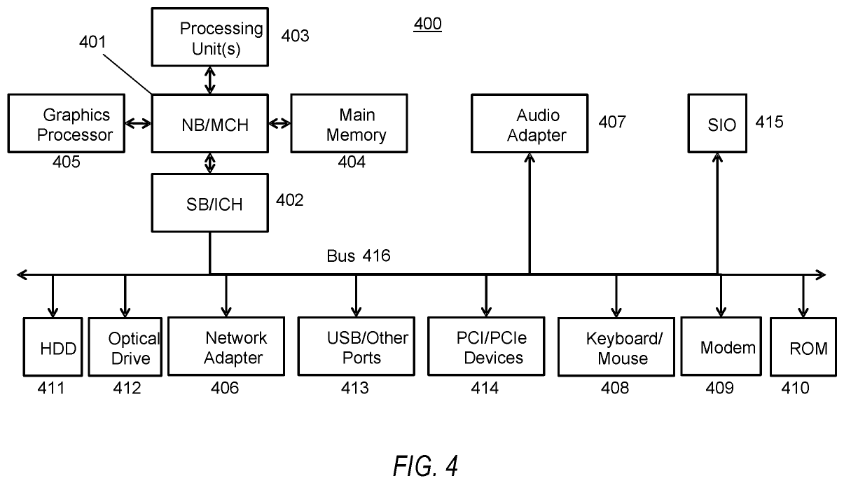

FIG. 4 is a block diagram of an example data processing system 400 in which aspects of the illustrative embodiments may be implemented. The data processing system 400 is an example of a computer, such as a server or client, in which computer usable code or instructions implementing the process for illustrative embodiments of the present invention are located. In one embodiment, FIG. 4 may represent a server computing device.

In the depicted example, data processing system 400 may employ a hub architecture including a north bridge and memory controller hub (NB/MCH) 401 and south bridge and input/output (I/O) controller hub (SB/ICH) 402. Processing unit 403, main memory 404, and graphics processor 405 may be connected to the NB/MCH 401. Graphics processor 405 may be connected to the NB/MCH 401 through an accelerated graphics port (AGP) (not shown in FIG. 4).

In the depicted example, the network adapter 406 connects to the SB/ICH 402. The audio adapter 407, keyboard and mouse adapter 408, modem 409, read only memory (ROM) 410, hard disk drive (HDD) 411, optical drive (CD or DVD) 412, universal serial bus (USB) ports and other communication ports 413, and the PCI/PCIe devices 414 may connect to the SB/ICH 402 through bus system 416. PCI/PCIe devices 414 may include Ethernet adapters, add-in cards, and PC cards for notebook computers. ROM 410 may be, for example, a flash basic input/output system (BIOS). The HDD 411 and optical drive 412 may use an integrated drive electronics (IDE) or serial advanced technology attachment (SATA) interface. The super I/O (SIO) device 415 may be connected to the SB/ICH 402.

An operating system may run on processing unit 403. The operating system could coordinate and provide control of various components within the data processing system 400. As a client, the operating system may be a commercially available operating system. An object-oriented programming system, such as the Java™ programming system, may run in conjunction with the operating system and provide calls to the operating system from the object-oriented programs or applications executing on data processing system 400. As a server, the data processing system 400 may be an IBM® eServer™ System P® running the Advanced Interactive Executive operating system or the Linux operating system. The data processing system 400 may be a symmetric multiprocessor (SMP) system that may include a plurality of processors in the processing unit 403. Alternatively, a single processor system may be employed.

Instructions for the operating system, the object-oriented programming system, and applications or programs are located on storage devices, such as the HDD 411, and are loaded into the main memory 404 for execution by the processing unit 403. The processes for embodiments of the generation system may be performed by the processing unit 403 using computer usable program code, which may be located in a memory such as, for example, main memory 404, ROM 410, or in one or more peripheral devices.

A bus system 416 may be comprised of one or more busses. The bus system 416 may be implemented using any type of communication fabric or architecture that may provide for a transfer of data between different components or devices attached to the fabric or architecture. A communication unit such as the modem 409 or network adapter 406 may include one or more devices that may be used to transmit and receive data.

Those of ordinary skill in the art will appreciate that the hardware depicted in FIG. 4 may vary depending on the implementation. Other internal hardware or peripheral devices, such as flash memory, equivalent non-volatile memory, or optical disk drives may be used in addition to or in place of the hardware depicted. Moreover, the data processing system 400 may take the form of a number of different data processing systems, including but not limited to, client computing devices, server computing devices, tablet computers, laptop computers, telephone or other communication devices, personal digital assistants, and the like. Essentially, the data processing system 400 may be any known or later developed data processing system without architectural limitation.

The computer readable storage medium may be a tangible device that may retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a head disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein may be downloaded to respective computing/processing devices from a computer readable storage medium or to an external computer or external storage device via a network, for example, the Internet, a local area network (LAN), a wide area network (WAN), and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on a remote computer, or entirely on the remote computer or server. In the latter scenario, the remote computer may be connected to the user's computer through any type of network, including LAN or WAN, or the connection may be made to an external computer (for example, through the Internet using an Internet Service Provider.) In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, may be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that may direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operation steps to be performed on the computer, other programmable apparatus, or other device to produce a computer implemented process, such that the instructions which execute on the computer, other programmable apparatus, or other device implement the functions/acts specified in the flowchart and/or block diagram block(s).

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical functions. In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts or carry out combinations of special purpose hardware and computer instructions.

The present description and claims may make use of the terms “a,” “at least one of,” and “one or more of,” with regard to particular features and elements of the illustrative embodiments. It should be appreciated that these terms and phrases are intended to state that there is at least one of the particular feature or element present in the particular illustrative embodiment, but that more than one may also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art, in view of the present description, that there are many other alternative implementations for these various elements that may be utilized in addition to, or in replacement of, the examples provided herein without departing from the spirit and scope of the present invention.

The system and processes of the figures are not exclusive. Other systems, processes, and menus may be derived in accordance with the principles of embodiments described herein to accomplish the same objectives. It is to be understood that the embodiments and variations shown and described herein are for illustration purposes only. Modifications to the current design may be implemented by those skilled in the art, without departing from the scope of the embodiments. As described herein, the various systems, subsystems, agents, managers and processes may be implemented using hardware components, software components, and/or combinations thereof. No claim element herein is to be construed under the provisions of 35 U.S.C. 112 (f) unless the element is expressly recited using the phrase “means for.”

Although the invention has been described with reference to exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as fall within the true spirit and scope of the invention.

What is claimed is:

1. A computer implemented method in a data processing system comprising a processor and a memory comprising instructions, which are executed by the processor to cause the processor to implement the method of identifying a group of medical professionals for performing an endoscopic procedure on a particular patient, the method comprising:
training, by the processor, a machine learning model with a plurality of electronic medical records of different patients having a history of one or more endoscopic procedures, wherein each electronic medical record includes a group of medical professionals performing the one or more endoscopic procedures, and recovery time of each endoscopic procedure, wherein the group of medical professionals include at least one physician, at least one technician, and at least one nurse;
receiving, by the machine learning model, an electronic medical record of a new patient intending to have an endoscopic procedure;
calculating, by the machine learning model, a score for each medical professional in a medical organization representing a level of match between the new patient and each medical professional, wherein all the medical professionals in the medical organization are divided into three categories of physician, technician, and nurse; and
identifying, by the machine learning model, a group of medical professionals for performing the endoscopic procedure on the new patient, wherein the group of medical professionals include a physician having the highest score in a category of physician, a technician having the highest score in a category of technician, and a nurse having the highest score in a category of nurse.

2. The method as recited in claim 1, further comprising:
calculating, by the machine learning model, the score through one or more of machine learning algorithms including logistic regression, Bipartite Ranking, k-partite Ranking, Ranking with Real-Valued Labels, General Instance Ranking, Ranking SVM, RankBoost, and RankNet.

3. The method as recited in claim 1, further comprising:
if there was a prior patient having short recovery time after the one or more endoscopic procedures performed by the group of the medical professionals including more than one nurse, and the prior patient has substantially the same medical condition as that of the new patient,
adding, by the machine learning model, an additional nurse into the group of medical professionals for performing the endoscopic procedure on the new patient.

4. The method as recited in claim 1, further comprising:
if there was a prior patient having short recovery time after the one or more endoscopic procedures performed by the group of the medical professionals including more than one technician, and the prior patient has substantially the same medical condition as that of the new patient,
adding, by the machine learning model, an additional technician into the group of medical professionals for performing the endoscopic procedure on the new patient.

5. The method as recited in claim 1, further comprising:
assigning, by the machine learning model, a more experienced group of medical professionals if the new patient has a plurality of diseases or at least one comorbidity.

6. The method as recited in claim 5, wherein the plurality of diseases includes at least two chronic conditions including type-2 diabetes, hypertension, hyperlipidemia, arthritis, and depression.

7. The method as recited in claim 1, wherein each electronic medical record includes at least one of characteristics including demographic details, allergies, diagnoses, vital signs, laboratory tests, clinical narrative notes, regular physical exams, pathology reports, discharge summaries, radiology reports, cardiology reports, encounters, comorbidities, endoscopic procedures, other procedures, and medications.

8. A computer program product for identifying a group of medical professionals for performing an endoscopic procedure on a particular patient, the computer program product comprising a computer readable storage medium having program instructions embodied therewith, the program instructions executable by a processor to cause the processor to:
train a machine learning model with a plurality of electronic medical records of different patients having a history of one or more endoscopic procedures, wherein each electronic medical record includes a group of medical professionals performing the one or more endoscopic procedures, and recovery time of each endoscopic procedure, wherein the group of medical professionals include at least one physician, at least one technician, and at least one nurse;
receive an electronic medical record of a new patient intending to have an endoscopic procedure;
calculate a score for each medical professional in a medical organization representing a level of match between the new patient and each medical professional, wherein all the medical professionals in the medical organization are divided into three categories of physician, technician, and nurse; and
identify a group of medical professionals for performing the endoscopic procedure on the new patient, wherein the group of medical professionals includes a physician having the highest score in a category of physician, a technician having the highest score in a category of technician, and a nurse having the highest score in a category of nurse.

9. The computer program product as recited in claim 8, wherein the processor is further caused to:
calculate the score through one or more of machine learning algorithms including logistic regression, Bipartite Ranking, k-partite Ranking, Ranking with Real-Valued Labels, General Instance Ranking, Ranking SVM, RankBoost, and RankNet.

10. The computer program product as recited in claim 8, wherein the processor is further caused to:
if there was a prior patient having short recovery time after the one or more endoscopic procedures performed by the group of the medical professionals including more than one nurse, and the prior patient has substantially the same medical condition as that of the new patient,
add an additional nurse into the group of medical professionals for performing the endoscopic procedure on the new patient.

11. The computer program product as recited in claim 8, wherein the processor is further caused to:
if there was a prior patient having short recovery time after the one or more endoscopic procedures performed by the group of the medical professionals including more than one technician, and the prior patient has substantially the same medical condition as that of the new patient,
add an additional technician into the group of medical professionals for performing the endoscopic procedure on the new patient.

12. The computer program product as recited in claim 8, wherein the processor is further caused to:
assign a more experienced group of medical professionals if the new patient has a plurality of diseases or at least one comorbidity.

13. The computer program product as recited in claim 12, wherein the plurality of diseases includes at least two chronic conditions including type-2 diabetes, hypertension, hyperlipidemia, arthritis, and depression.

14. The computer program product as recited in claim 8, wherein each electronic medical record includes at least one of characteristics including demographic details, allergies, diagnoses, vital signs, laboratory tests, clinical narrative notes, regular physical exams, pathology reports, discharge summaries, radiology reports, cardiology reports, encounters, comorbidities, endoscopic procedures, other procedures, and medications.

15. A system for identifying a group of medical professionals for performing an endoscopic procedure on a particular patient, comprising:
a processor configured to:
train a machine learning model with a plurality of electronic medical records of different patients having a history of one or more endoscopic procedures, wherein each electronic medical record includes a group of medical professionals performing the one or more endoscopic procedures, and recovery time of each endoscopic procedure, wherein the group of medical professionals include at least one physician, at least one technician, and at least one nurse;
receive an electronic medical record of a new patient intending to have an endoscopic procedure;
calculate a score for each medical professional in a medical organization representing a level of match between the new patient and each medical professional, wherein all the medical professionals in the medical organization are divided into three categories of physician, technician, and nurse; and
identify a group of medical professionals for performing the endoscopic procedure on the new patient, wherein the group of medical professionals include a physician having the highest score in a category of physician, a technician having the highest score in a category of technician, and a nurse having the highest score in a category of nurse.

16. The system as recited in claim 15, wherein the processor is further configured to:
calculate the score through one or more of machine learning algorithms including logistic regression, Bipartite Ranking, k-partite Ranking, Ranking with Real-Valued Labels, General Instance Ranking, Ranking SVM, RankBoost, and RankNet.

17. The system as recited in claim 16, wherein the processor is further configured to:
if there was a prior patient having short recovery time after the one or more endoscopic procedures performed by the group of the medical professionals including more than one nurse, and the prior patient has substantially the same medical condition as that of the new patient,
add an additional nurse into the group of medical professionals for performing the endoscopic procedure on the new patient.

18. The system as recited in claim 16, wherein the processor is further configured to:
if there was a prior patient having short recovery time after the one or more endoscopic procedures performed by the group of the medical professionals including more than one technician, and the prior patient has substantially the same medical condition as that of the new patient,
add an additional technician into the group of medical professionals for performing the endoscopic procedure on the new patient.

19. The system as recited in claim 17, wherein the processor is further configured to:
assign a more experienced group of medical professionals if the new patient has a plurality of diseases or at least one comorbidity.

20. The system as recited in claim 19, wherein the plurality of diseases includes at least two chronic conditions including type-2 diabetes, hypertension, hyperlipidemia, arthritis, and depression.
